(19) United States
(12) Patent Application Publication
(10) Pub. No.: US 2020/0129080 A1
(43) Pub. Date: Apr. 30, 2020

(54) SYSTEMS AND METHODS FOR PERFORMING BIOMETRIC AUTHENTICATION

(71) Applicant: BOARD OF SUPERVISORS OF LOUISIANA STATE UNIVERSITY AND AGRICULTURAL AND MECHANICAL COLLEGE, Baton Rouge, LA (US)

(72) Inventors: Limeng Pu, Baton Rouge, LA (US); Pedro Chacon Dominguez, Baton Rouge, LA (US); Hsiao-Chun Wu, Baton Rouge, LA (US); Jin-Woo Choi, Baton Rouge, LA (US)

(21) Appl. No.: 16/669,877

(22) Filed: Oct. 31, 2019

Related U.S. Application Data
(60) Provisional application No. 62/753,581, filed on Oct. 31, 2018.

(51) Int. Cl. A61B 5/024; A61B 5/0402; A61B 5/1455; A61B 5/00; G06F 21/32

(52) U.S. Cl. CPC A61B 5/02416; A61B 5/0402; G06F 21/32; A61B 5/0077; A61B 5/7278; A61B 5/14552

(57) ABSTRACT

A biometric authentication system and method are provided that are based on PPG signals acquired using non-invasive methods, such as pulse oximeter, for example. Compared to the other biometric authentication approaches, PPG signals are relatively easy to acquire, relatively difficult to duplicate, and can be adaptively updated, which are qualities that make them well suited for biometric authentication. However, PPG signals are often corrupted by motion artifacts (MAs) due to the unique measurement technique, i.e., placement of a sensor close to the skin. The system and method employ an MA removal algorithm to remove or mitigate MAs to provide a PPG-based biometric authentication solution that is robust and that can be implemented relatively easily and cost effectively.

### CROSS-REFERENCE TO RELATED APPLICATIONS

This application is a U.S. nonprovisional application that claims priority to, and the benefit of the filing date of, U.S provisional application Ser. No. 62/753,581, filed on Oct. 31, 2018, entitled “SYSTEMS AND METHODS FOR PERFORMING BIOMETRIC AUTHENTICATION,” which is hereby incorporated by reference herein in its entirety.

### TECHNICAL FIELD

The present invention generally relates to biometrics, and more particularly, to systems and methods for performing biometric authentication.

### BACKGROUND

A variety of approaches exist for performing biometric authentication, which is authentication based on images of human biometric features, such as fingerprints, the iris and palm veins, for example. Each of the known approaches has distinct advantages, but also significant defects. Authentication methods based on fingerprint images are the most widely adopted methods of biometric authentication, and are employed in most smart phones. Such methods utilize scanned images of fingerprints, which can be acquired using many different types of equipment, and features that are extracted from the scanned images. The extracted features are encoded into unique sequences for different users.

One problem associated with fingerprint authentication systems is that, because they are based on image recognition, live-tissue verification is a major drawback. In other words, with only a small amount of tampering to the system, one can use an image (e.g., a photograph) of an authorized user's fingerprint for the authentication procedure. There is no way for the system to distinguish between a real fingerprint and an image of the fingerprint. The second problem is that fingerprints are easy to duplicate. Software exists that can extract fingerprints from an image of the finger acquired from a particular viewpoint. This largely undermines the security of the authentication system. Another problem is that the system may not recognize the authorized user's fingerprint if the finger is wet or stained, which can lead to the need for regular submissions of new data.

Iris image authentication systems and methods are very accurate compared to other biometric authentication systems and methods. Such systems and methods involve the use of specific feature extraction algorithms to extract features from images of the iris. During the authentication process, unique patterns in a scanned iris image are compared to features extracted from the authorized user's iris. One of the problems associated with such systems is that the processing time for the comparison is longer than that of other authentication methods due to the complex nature of the image feature patterns. Another problem associated with such systems is that they are very expensive. Yet another problem associated with such systems is the aforementioned live-tissue verification problem, i.e., the system cannot discern the difference between a real iris and an image of an iris.

Palm vein image authentication is a relatively new type of biometric authentication. The image is captured using a near-infrared (NIR) camera. The features are extracted from the acquired images and then compared to the stored features of the authorized users. The main problem with this approach is the aforementioned live-tissue verification problem.

Biometric authentication techniques based on photoplethysmogram (PPG) signals have been proposed for many years. PPG signals are signals acquired using non-invasive methods such as pulse oximetry. A pulse oximeter illuminates the skin and measures a change in light absorption between the emitted light and the reflected light. Compared to the other biometric authentication approaches described above, PPG signals are relatively easy to acquire, relatively difficult to duplicate, and can be adaptively updated. These qualities make PPG signals well suited for biometric authentication. However, most of the proposed PPG authentication techniques utilize fiducial features, which are time-domain-based features, resulting in performance that is less than ideal and not very robust.

A need exists for a biometric authentication system that overcomes the problems associated with the known biometric authentication methods and systems.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a block diagram of the PPG signal-based authentication system in accordance with a representative embodiment.

FIG. 2 is a plot of signal amplitude as a function of number of samples that shows an example of detected peaks 12-22 of a segment of a signal.

FIG. 3 is a plot of signal amplitude as a function of number of samples for a plurality of templates obtained for user k (top portion) and a plot of the ensemble averaged template for user k (bottom portion).

FIGS. 4A and 4B graphically illustrate the scaling functions at time t=0.5 and t=1.0, respectively, and FIGS. 4C and 4D graphically illustrate the wavelet functions of GHM multiwavelets at t=0.5 and 1.0, respectively.

FIG. 5 is a block diagram of an example of a level 2 discrete multiwavelet decomposition process represented by the multiwavelet decomposition block shown in FIG. 1.

FIG. 6 is a block diagram of an example of the MLP block shown in FIG. 1 with one hidden layer.

FIG. 7 is a graph of FRR/FAR as a function of threshold value that shows the results for the average FAR/FRR plot over 100 random epochs.

FIG. 8 is a graph of false positive rate vs. true positive rate and shows the receiver operation characteristics (ROC) curve for the user identification over 100 epochs.

FIG. 9 is a graph of the identification accuracy vs. training signal length for the system 1 shown in FIG. 1.

FIG. 10 is a flow diagram that illustrates the method for performing biometric authentication in accordance with a representative embodiment.

FIG. 11 is a flow diagram that illustrates the steps represented by block 102 of FIG. 10 in accordance with a representative embodiment.

### DETAILED DESCRIPTION

Representative embodiments described herein are directed to a biometric authentication system and method that are based on PPG signals acquired using non-invasive methods, such as pulse oximetry, for example. As indicated above, compared to the other biometric authentication approaches described above, PPG signals are relatively easy to acquire, relatively difficult to duplicate, and can be adaptively updated, which are qualities that make them well suited for biometric authentication. However, PPG signals are often corrupted by motion artifacts (MAs) due to the unique measurement technique, i.e., placement of a sensor close to the skin. Embodiments described herein overcome the MA issue to provide a PPG-based biometric authentication solution that is robust and that can be implemented relatively easily and cost effectively.

In accordance with a representative embodiment, PPG signals are acquired, filtered by a relatively simple filtering algorithm and then processed by an MA removal algorithm that tailors the PPG signals to remove MA from the PPG signals prior to using them for authentication.

In accordance with a representative embodiment, after the MA removal algorithm has been performed, a number of templates of the same length are extracted for each individual, or subject, to be authenticated. The extracted templates for each individual are then combined into one single template, which results in one template for each authorized user. Subsequent to generating the templates, a multiwavelet decomposition algorithm is performed on the templates to convert them into a sequence of feature numbers. The feature numbers are used as an input for a machine learning architecture. The training data for each individual is measured in a relatively short period of time, while the testing data is measured in an even shorter period of time, which makes it comparable in authentication speed with other biometric systems and methods.

Experiments were carried out using publicly-available data on forty-two subjects and using data on three subjects measured by the inventors with an oximeter. The overall performance was found to be comparable to other biometric authentication systems and superb (less than 1% of false rejection and false acceptance rate) compared to the existing methods and systems for PPG-based authentication in terms of both the accuracy of the authentication and the length of data required.

Besides the relatively ease of implementation and the robustness of the authentication system and method disclosed herein, PPG signals can be used to simultaneously achieve continuous authentication and health status monitoring. These features are not available with any other biometric authentication systems and methods.

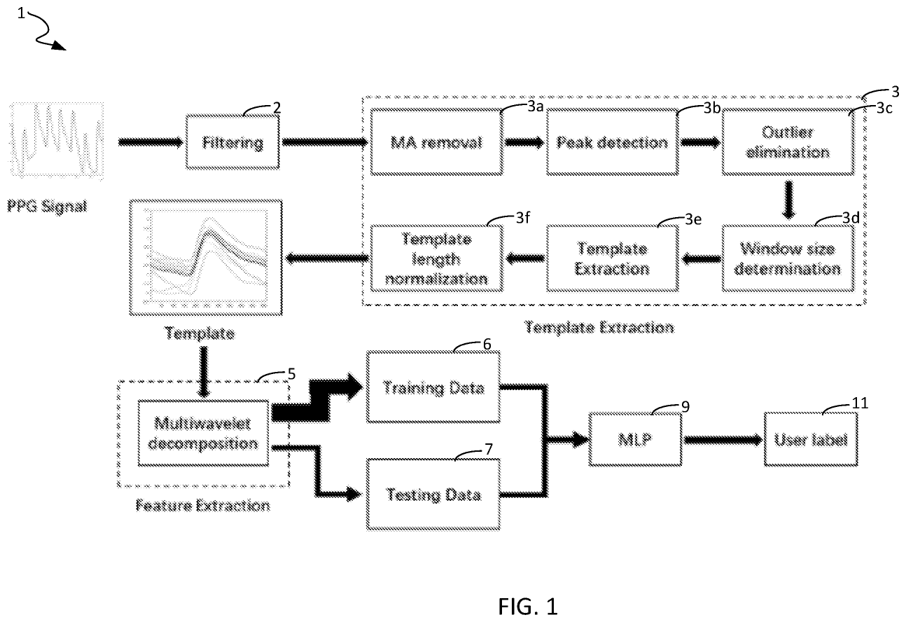

A few representative embodiments of the system and method that provide such an EA solution will now be described with reference to FIGS. 1-9, in which like reference numerals represent like components, elements or features. It should be noted that features, elements or components in the figures are not intended to be drawn to scale, emphasis being placed instead on demonstrating inventive principles and concepts. It should be noted that the inventive principles and concepts are not limited to the representative embodiments described herein, as will be understood by those of skill in the art in view of the description provided herein.

In the following detailed description, for purposes of explanation and not limitation, exemplary, or representative, embodiments disclosing specific details are set forth in order to provide a thorough understanding of inventive principles and concepts. However, it will be apparent to one of ordinary skill in the art having the benefit of the present disclosure that other embodiments according to the present teachings that are not explicitly described or shown herein are within the scope of the appended claims. Moreover, descriptions of well-known apparatuses and methods may be omitted so as not to obscure the description of the exemplary embodiments. Such methods and apparatuses are clearly within the scope of the present teachings, as will be understood by those of skill in the art. It should also be understood that the word “example,” as used herein, is intended to be non-exclusionary and non-limiting in nature.

The terminology used herein is for purposes of describing particular embodiments only, and is not intended to be limiting. The defined terms are in addition to the technical, scientific, or ordinary meanings of the defined terms as commonly understood and accepted in the relevant context.

The terms “a,” “an” and “the” include both singular and plural referents, unless the context clearly dictates otherwise. Thus, for example, “a device” includes one device and plural devices. The terms “substantial” or “substantially” mean to within acceptable limits or degrees acceptable to those of skill in the art. For example, the term “substantially parallel to” means that a structure or device may not be made perfectly parallel to some other structure or device due to tolerances or imperfections in the process by which the structures or devices are made. The term “approximately” means to within an acceptable limit or amount to one of ordinary skill in the art. Relative terms, such as “over,” “above,” “below,” “top,” “bottom,” “upper” and “lower” may be used to describe the various elements' relationships to one another, as illustrated in the accompanying drawings. These relative terms are intended to encompass different orientations of the device and/or elements in addition to the orientation depicted in the drawings. For example, if the device were inverted with respect to the view in the drawings, an element described as “above” another element, for example, would now be below that element.

Relative terms may be used to describe the various elements' relationships to one another, as illustrated in the accompanying drawings. These relative terms are intended to encompass different orientations of the device and/or elements in addition to the orientation depicted in the drawings.

The term “memory” or “memory device”, as those terms are used herein, are intended to denote a non-transitory computer-readable storage medium that is capable of storing computer instructions, or computer code, for execution by one or more processors. References herein to “memory” or “memory device” should be interpreted as one or more memories or memory devices. The memory may, for example, be multiple memories within the same computer system. The memory may also be multiple memories distributed amongst multiple computer systems or computing devices.

A “processor,” “processing device,” or “processing logic,” as those terms are used herein, encompass at least one electronic device that is configured to perform one or more processing algorithms that process signals. The electronic device(s) may perform the algorithm(s) in hardware, software or firmware, or a combination thereof. References herein to a system comprising “a processor” or “a processing device” or “processing logic” should be interpreted as one or more processors or processing cores. The processor may, for instance, be a multi-core processor. A processor may also refer to a collection of processors within a single computer system or distributed amongst multiple computer systems. The term “computer,” as that term is used herein, should be interpreted as possibly referring to a single computer or computing device or to a collection or network of computers or computing devices, each comprising a processor or processors. Instructions of a computer program can be performed by a single computer or processor or by multiple processors that may be within the same computer or that may be distributed across multiple computers.

Because motion artifacts (MAs) can cause serious problems in PPG signal processing, the detection and elimination (or mitigation) of MAs are important when PPG signals are used to measure biological information. In accordance with a representative embodiment, the method includes an MA detection method having three major stages: (i) optimal window-size determination, (ii) short-time variance extraction, and (iii) transition detection.

Usually the PPG signal waveforms appear to be very different across subjects. Therefore, using a subject-independent window-size (constant short-time window-length) is non-optimal when one needs to subsequently acquire robust variance features. In fact, the optimal window-size should be “subject-dependent,” which means it should depend on the individual subject's signal characteristics. In accordance with a representative embodiment, an unsupervised (no training is necessary) subject-dependent window-size determination technique is used to search for the “optimal” window-size. In accordance with a representative embodiment, a robustness feature, referred to herein as a short-time variance, is utilized for MA detection because the associated computational burden is very light. The transition points (at which there appears to be a change from the regular signal to the motion artifact, or vice versa) can thus be located by comparing the short-time variances with an appropriate threshold.

Once the artifact intervals are spotted accordingly, the corresponding artifact signal data can be tailored (mitigated or eliminated completely from the original (raw) signal waveform by employing the aforementioned MA removal algorithm. In accordance with a representative embodiment, the MA removal algorithm comprises the following. The short-time variance is the underlying feature for motion-artifact detection. The short-time variance, which is a function of window-size, M, can be formulated as the result from a mapping from the original PPG signal sequence x(n), n=0, 1, 2, . . . :

x(n) [symbol] γ_{μ}^{M}(m), m=0, 1, 2, . . . , (1)

where

$${ϒ}_{μ}^{M} ( m ) \frac{1}{M - 1} {\sum }_{n = m μ}^{m μ + M - 1} {[ x ( n ) - {\overline{x}}_{μ}^{M} ( m ) ]}^{2} , \qquad ( 2 )$$

n denotes the original signal sample index, m denotes the sliding-window index, μ denotes the window forward size, and

$${\overline{x}}_{μ}^{M} ( m ) \frac{1}{M} {\sum }_{n = m μ}^{m μ + M - 1} x ( n ) . \qquad ( 3 )$$

The manner in which the optimal window size M may be determined will be presented next. According to previous experience of the inventors in ultrasonic signal processing, a nonlinear program can be facilitated to find the optimal window size M. The “smoothness” of the variance sequence γ_{μ}^{M}(m) is the objective function while the “compact-support” requirement corresponds to the nonlinear constraint. The compact-support requirement implies a steep-transitioned variance sequence γ_{μ}^{M}(m). A kurtosis function ϰ[γ_{μ}^{M}(m)] can be used to define such a constrained optimization problem. Define

$$\begin{matrix}{p}_{m} \frac{{ϒ}_{μ}^{M} ( m )}{{\sum }_{q} {ϒ}_{μ}^{M} ( q )} , & ( 4 ) \\ {\sum }_{m} [ ( m - 1 ) μ + 1 ] {p}_{m} , & ( 5 ) \\ {k}_{} {\sum }_{m} {[ ( m - 1 ) μ + 1 - ]}^{k} {p}_{m} , k = 2 , 4. & ( 6 )\end{matrix}$$

The kurtosis of the short-time variances γ_{μ}^{M}(m) can be formulated as:

ϰ[γ_{μ}^{M}(m)] [symbol] [symbol] ₄/ [symbol] ₂². (7)

Note that pₘ sequence in Eq. (4) can satisfy the probability axioms. It has been proved that ϰ[γ_{μ}^{M}(m)] is μ-multiple-shift invariant. That is, one can start a sliding window to sample data at any time instant and the optimal window-size will remain the same. In other words, the present method is resilient against the onset ambiguity.

Define the kurtosis-sensitivity with respect to M as

$$S ( M ) {max}_{m} \frac{\vert {ϒ}_{μ}^{M} ( m ) - {ϒ}_{μ}^{2 M} ( m ) \vert}{{ϒ}_{μ}^{M} ( m )} . \qquad ( 8 )$$

According to Eqs. (1)-(8), the optimal window-size M can thus be determined from the following nonlinear program:

M^{opt}=max(M)

subject to

S(M)< [symbol] , (9)

where [symbol] is a pre-set upper-bound for the kurtosis-sensitivity constraint function. After the optimal window-size is determined according to Eq. (9), the detection of motion artifacts can take place thereby. After the detection of segments pertinent to motion artifacts is carried out throughout the entire PPG signal sequence, we denote the collection of the detected transition-points (the occasions of the transitions from the regular signal to a motion artifact and vice versa) by {[t₁, τ₁], [t₂, τ₂], . . . , [tᵢ, τᵢ], . . . }. Thus, we can represent the i^{th} detected motion-artifact signal segment Ω(n) by

$${Ω}_{i} ( n ) \{ \begin{matrix}x ( n ) , & {t}_{i} \leq n \leq {τ}_{i} , \\ 0 , & elsewhere\end{matrix} . \qquad ( 10 )$$

Note that when Ωᵢ(n)=x(n), the corresponding signal samples carry “useless” information (or no measurement should be taken at the corresponding time instant n). Therefore, the “tailored” signal sequence x′(n) can be expressed as

x′(n) [symbol] x(n)−ΣᵢΩᵢ(n). (11)

If we convert (tailor) x(n) to x′(n), the original waveform of the PPG signal can be preserved, but other existing spectrum-based methods fail to do so. When x′(n)=0, the PPG signal is tailored or eliminated, such zero-valued signal samples x′(n) should be thrown out without any further use. That is, one should “stop” sampling the motion-artifact segments because no accurate information can be extracted from those data. Henceforth, the tailored signal sequence x′(n) can be further processed for the fast SpO₂ computation in addition to the heart-beat rate tracking and other relevant physiological information-retrieval.

### PPG Signal Based Authentication

FIG. 1 is a block diagram of the PPG signal-based authentication system 1 in accordance with a representative embodiment. The system 1 comprises a simple filter block 2, a template extraction block 3, a multiwavelet decomposition block 5, a training data block 6, a testing data block 7, an autoencoder block 9 and a user label block 11. The blocks 2-11 represent respective algorithms that may be performed in hardware, software, firmware, or a combination thereof. For illustrative purposes, it will be assumed that the algorithms are implemented in software, firmware, or a combination thereof, executed by one or more computers or processors. The computer code for such algorithms may be stored in one or more suitable non-transitory computer-readable mediums, such as solid state memory, magnetic storage, optical storage, or a combination thereof.

Sub-block 3a of block 3 represents the aforementioned MA removal algorithm. As indicated above, in accordance with this representative embodiment, after the MAs have been removed or at least mitigated in the manner described above, the signal is further processed to extract templates for the machine learning algorithm. In accordance with a representative embodiment, the template extraction process includes five parts, namely, peak detection 3b, outlier elimination 3c, window-size determination 3d, template extraction 3e and template length normalization 3f. It should be noted, however, that a variety of configurations other than what is shown in block 3 may be used to perform MA removal and template extraction, as will be understood by one of skill in the art in view of the description provided herein.

After the template extraction processing has been performed, a multiwavelet decomposition algorithm, represented by block 5, preferably is performed on the extracted templates to obtain extracted multiwavelet coefficients. The extracted multiwavelet coefficients are used as the input feature for an autoencoder, which is a multilayer perceptron (MLP) in accordance with a representative embodiment. It should be noted, however, that autoencoders other than an MLP are suitable for use in the system 1.

Denoting the set of all authorized users as [symbol] ={1, 2, 3, . . . , k, . . . , K} and the tailored signal for each authorized user as x^{k}(n), the ′ can be dropped from Eq. (11) for the simplicity of expression. In accordance with an embodiment, the peak detection algorithm 3b is performed using the derivative-based method, although a variety of peak detection algorithms may be used for this purpose. A peak for the tailored signal of authorized user k, denoted as pᵢ^{k}, i=1, 2, 3, . . . , is defined as the point where the sign of the derivative of the signal changes from positive to negative. The derivative-based method usually performs well when the noise level of the signal is relatively low. Otherwise, many spurious peaks may be found. PPG signals are less noisy compared to other real-life signals because the noise can be easily removed by a bandpass filter with passband frequency from 5 Hz to 40 Hz, which may be employed in the filtering block 2. Thus, derivative-based peak finding fits very well in this situation. Though the employed peak finding is rather accurate, in order to eliminate any possible outliers, an outlier elimination algorithm preferably is carried out by block 3c to remove the undesired peaks caused by various factors. An outlier is a spurious peak having a value that is significantly larger than the median absolute deviation (MAD), which is defined as:

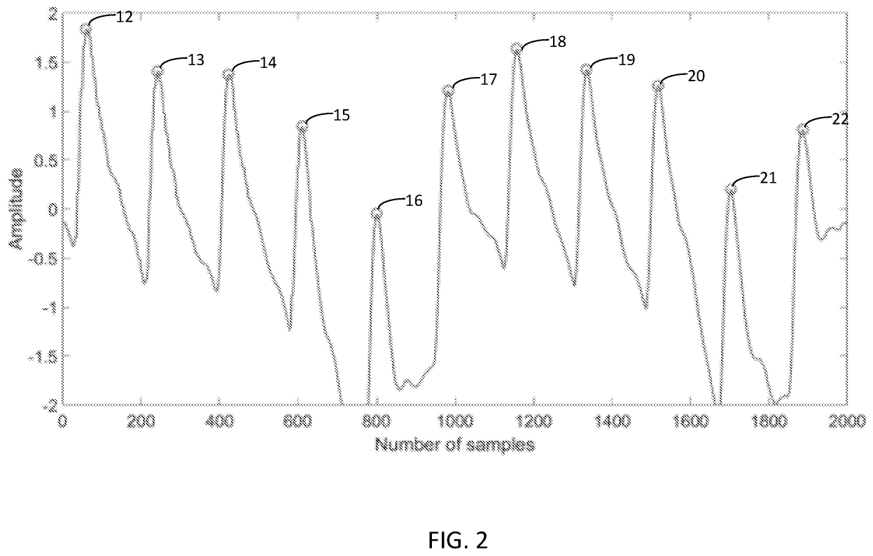

MAD=median(|ρᵢ^{k}−median(P^{k})|), i=1, 2, 3 . . . , p, (12)

where P^{k}={ρ₁^{k}, ρ₂^{k}, . . . , ρₚ^{k}} and p is the total number of peaks. FIG. 2 is a plot of signal amplitude as a function of number of samples that shows an example of detected peaks 12-22 of a segment of a signal.

After the true peaks are detected, one needs to extract the template, which is the signal segment that contains each pulsatile waveform. The extraction window size for authorized user k, W^{k}, is calculated as the average of the difference between successive peaks, which can be expressed as follows

$${Δ}^{k} = ⌈ \frac{1}{p} {\sum }_{i = 1}^{p} ( {ρ}_{i + 1}^{k} - {ρ}_{i}^{k} ) ⌉ , i = 1 , 2 , … , p , \qquad ( 13 )$$

where ┌·┐ is the round up operation. Since the window size Δ^{k} varies from individual to individual, the templates are normalized by interpolation (sub-block 3f) to have a uniform input for the multiwavelet feature extraction. A linear interpolation strategy preferably is employed here to normalize all the templates to have a length of 512.

Now one can extract many templates for authorized user k, denoted as [symbol] ^{k}={T₁^{k}, T₂^{k}, . . . , T_{M}^{k}}, from the corresponding tailored signal x^{k}(n). The ensemble average of all the templates is calculated using Eq. (14).

$${\tilde{T}}^{k} = \frac{1}{M} {\sum }_{j = 1}^{M} {T}_{j}^{k} \qquad ( 14 )$$

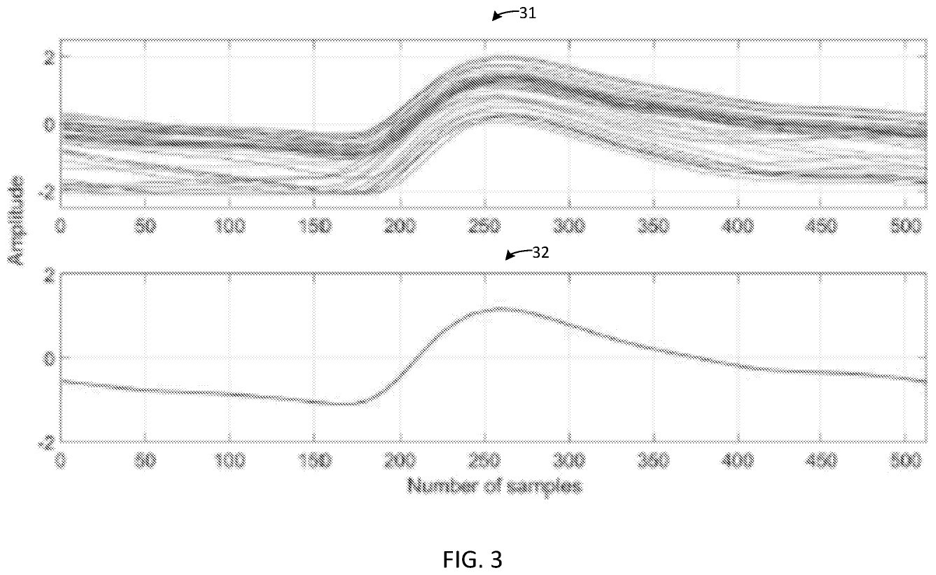

The ensemble averaged template \tilde{T}^{k} is used as the final input data for the multiwavelet feature extraction that is input to block 5 of FIG. 1. FIG. 3 shows a plot of signal amplitude as a function of number of samples for a plurality of templates obtained for user k (top portion 31) and a plot of the ensemble averaged template for user k (bottom portion 32). For exemplary purposes, all of the templates have been normalized to the length 512 by linear interpolation.

After the templates are successfully extracted, the multiwavelet decomposition algorithm 5 (FIG. 1) is employed to extract the features from the input ensemble averaged templates. Wavelet decomposition is a popular technique for feature extraction in biomedical signals since it is easy to implement and capable of providing good time-frequency information. In accordance with a representative embodiment, Geronimo-Hardin-Massopust (GHM) multiwavelets are used as the mother wavelets. Compared to conventional scalar wavelets, the multiwavelet system offers more superior performance in both approximation and reconstruction, compared to the conventional scalar wavelet systems. In the present case, the multiwavelet decomposition does not require the signal length to be power of 2 while it is a necessity in the wavelet decomposition. Besides, the multiwavelet decomposes the signal into two sets of coefficients, which provides more features and more refined approximation of the original signal.

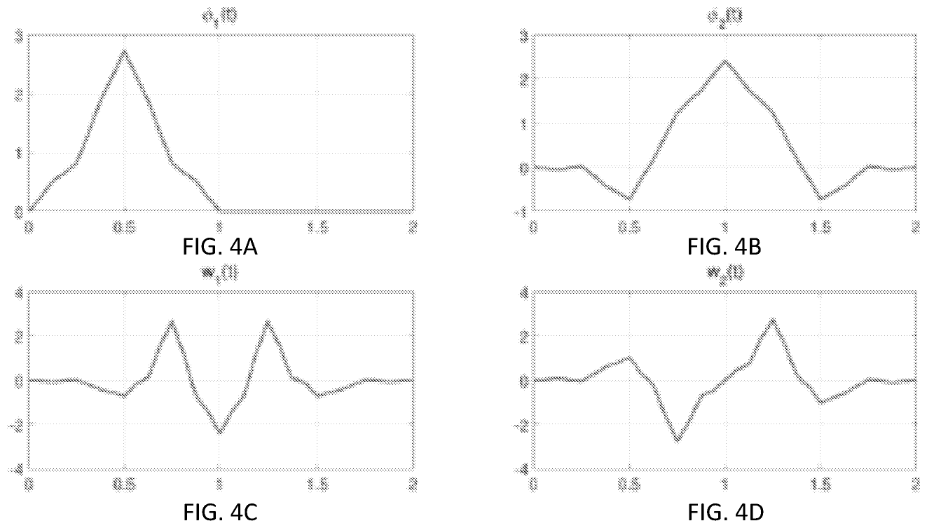

FIGS. 4A and 4B graphically illustrate the scaling functions at time t=0.5 and t=1.0, respectively, and FIGS. 4C and 4D graphically illustrate the wavelet functions of GHM multiwavelets at t=0.5 and 1.0, respectively. The scaling functions are denoted by ϕ and the wavelet functions are denoted by w. Similar to the scalar wavelet, the multiwavelets have to satisfy the dilation equations expressed as follow:

ϕ(t)=ΣₖC[k]ϕ(2t−k), (15)

W(t)=ΣₖD[k]ϕ(2t−k), (16)

where ϕ(t)=[ϕ₁(t), ϕ₂(t), . . . , ϕᵣ(t)] are the scaling function, W(t)=[w₁(t), . . . , wᵣ(t)] are the wavelet functions, and the r×r matrices C[k] and D[k] are the lowpass and highpass filters coefficients, respectively, for the multiwavelet filter banks, respectively.

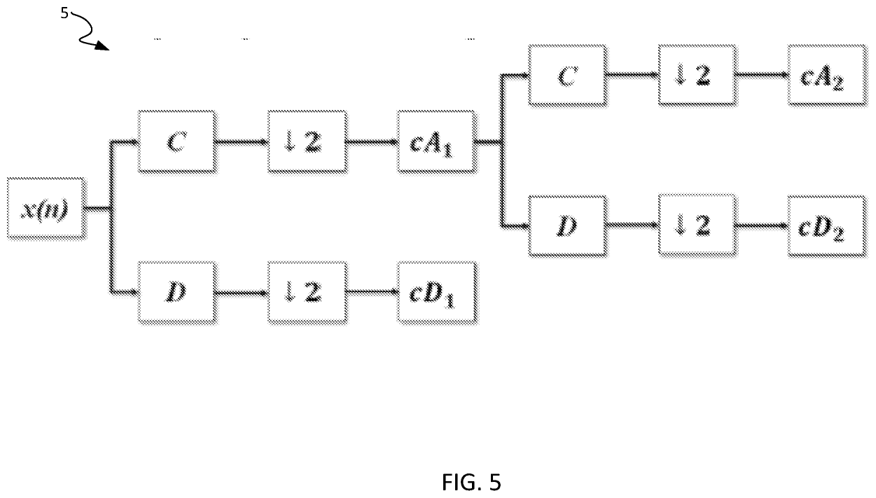

FIG. 5 is a block diagram of an example of a level 2 discrete multiwavelet decomposition process 5. In FIG. 5, C and D are the lowpass and highpass filter coefficients, respectively, cAⱼ is the level j approximation coefficient while cDⱼ is the level j detail coefficients. To get more decomposition levels, one can repeat the process with the approximation coefficients from the previous level. Since in the multiwavelet scenarios, C and D are matrices instead of scalars, the input signals are preprocessed. In accordance with a representative embodiment, repeat sampling is performed, which duplicates the input signal to be a N×r matrix, where N is the total length of the original input signal. The approximation and detail coefficients are the ultimate input for the MLP block 9 (FIG. 1).

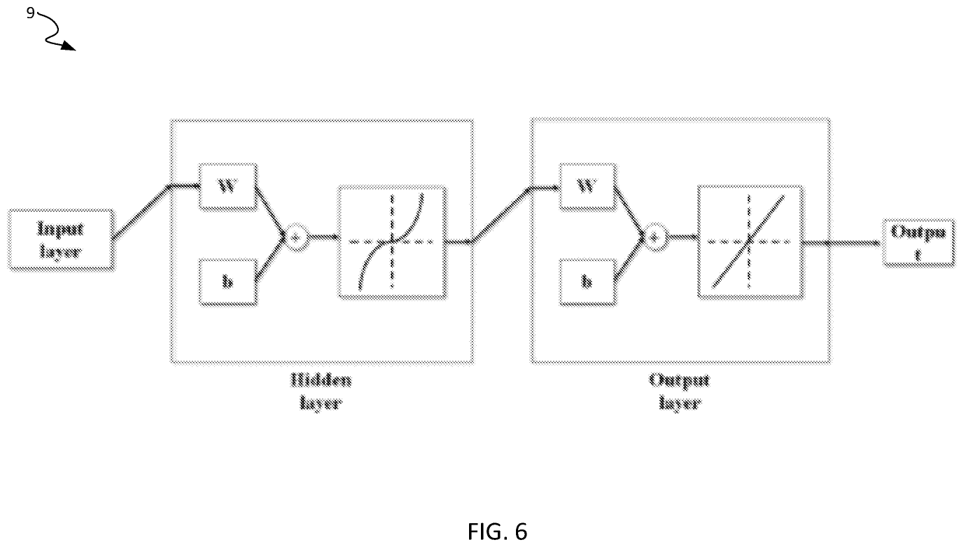

The MLP block 9 is used as the machine learning structure in this representative embodiment. It is a static feed forward neural network with one or more hidden layers. The input layer of the MLP block 9 contains the extracted features, which are the multiwavelet decomposition coefficients. The hidden/output layers contain sets of weights and bias and an activation function. The weights are updated by minimizing the cross-entropy. FIG. 6 is a block diagram of an example of the MLP block 9 with one hidden layer.

### Experiment Setup and Results

The dataset used to evaluate the performance of the system 1 shown in FIG. 1 and the associated method is the IEEE Transactions on Biomedical Engineering (TMBE) multiparameter respiratory rate dataset, which contains PPG signals of forty-two subjects from various ages and three subjects' data acquired with the system 1. Each of the PPG signals is eight minutes long and the sample rate used is 300 Hz. The dataset is first separated into authorized users and unauthorized users, where five subjects are assigned as authorized while the other forty are the unauthorized users. For the authorized users, 80% of the data is used for training while the other 20% is used as the testing set. Level 3 multiwavelet decomposition is employed. Each template will yield feature vectors with length of 1024.

The false rejection rate (FRR), false acceptance rate (FAR) and equal error rate (ERR), which is the point where FRR is equal to FAR, are used as the criterion for evaluating the performance of the authentication system 1. In this experiment, 100 random authorized/unauthorized split is adpoted and the final result is the average of all 100 epochs. The result is compared with the same strategy, but using Daubechies wavelet instead of the GHM multiwavelets.

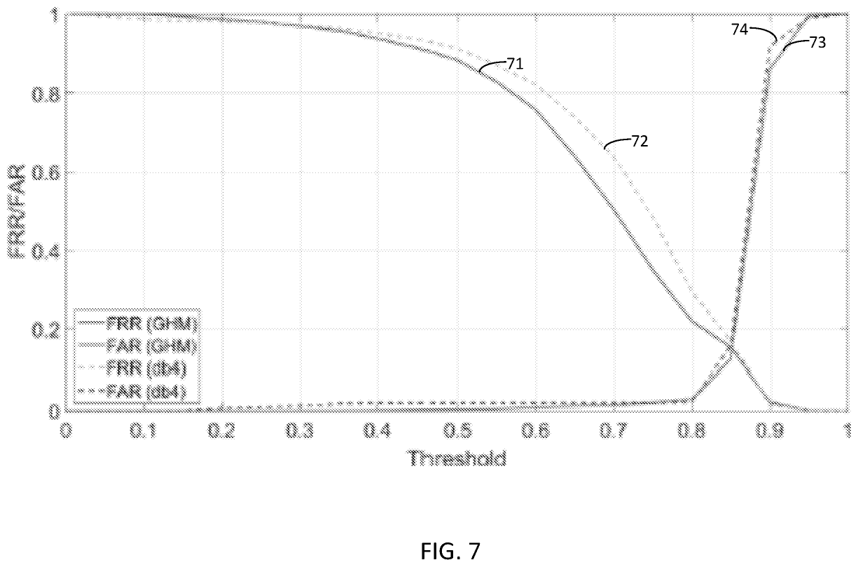

FIG. 7 is a graph of FRR/FAR as a function of threshold value that shows the results for the average FAR/FRR plot over 100 random epochs. The best EER is around 18%. Curves 71 and 72 correspond to the FRR GHM multiwavelet and Daubechies wavelet results, respectively. Curves 73 and 74 correspond to the FAR GHM multiwavelet and Daubechies wavelet results, respectively.

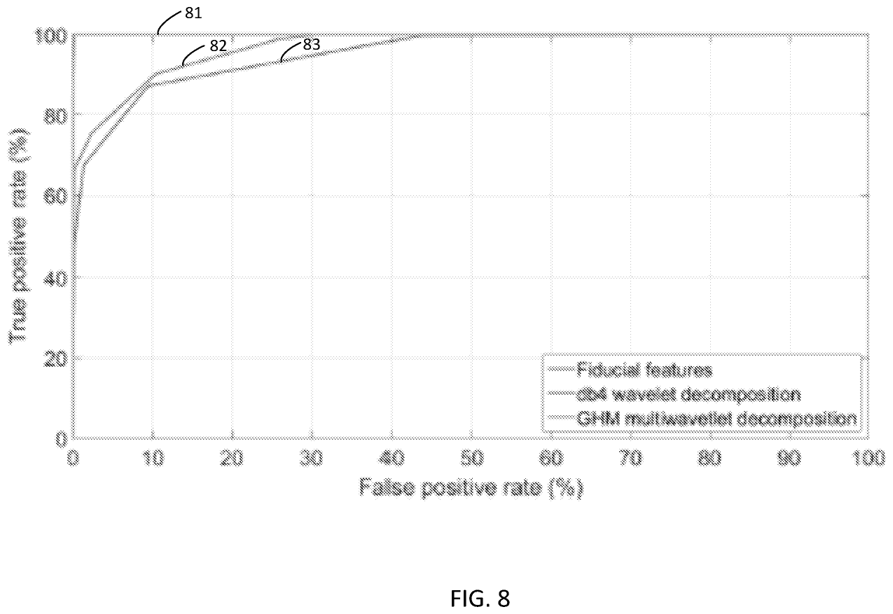

The system 1 can successfully recognize the authorized user from the unauthorized users. The next step is to identify each authorized user. FIG. 8 is a graph of false positive rate vs. true positive rate and shows the receiver operation characteristics (ROC) curve for the user identification over 100 epochs. Curves 81, 82 and 83 are the ROC curves for the GHM multiwavelet decomposition in accordance with an embodiment, the Daubechies wavelet decomposition and the known fiducial features method, respectively. FIG. 8 demonstrates the effectiveness of the system 1 and the method, which can reach almost 100% accuracy even with a small number of training data and even fewer segments for template averaging.

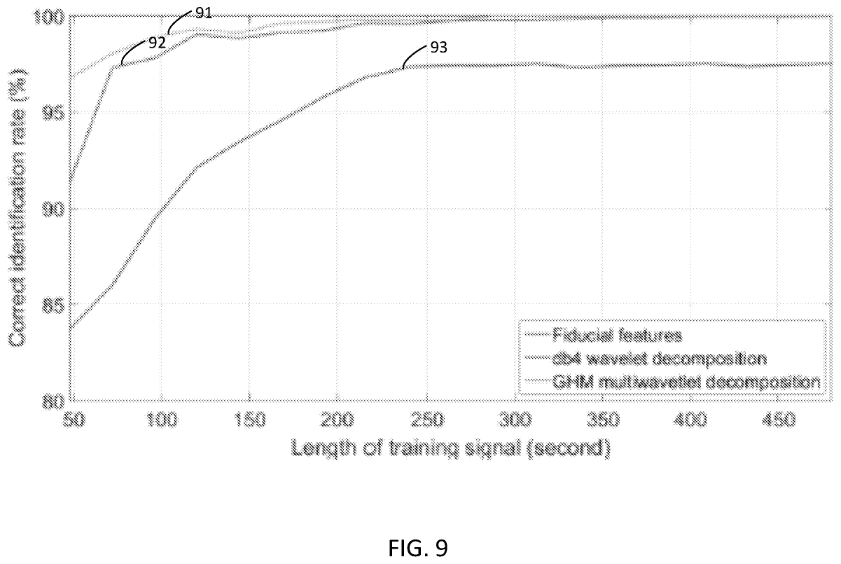

Another advantage of the system 1 is that it requires fewer samples for the training set to achieve acceptable identification results. FIG. 9 is a graph of the identification accuracy vs. training signal length for the system 1 shown in FIG. 1. Curves 91, 92 and 93 correspond the GHM multiwavelet decomposition approach of the exemplary embodiment, the Daubechies wavelet approach and the fiducial features approach, respectively. It can be seen from FIG. 9 that the method performed by system 1 greatly outperforms the best existing method when the training data length is less than 1 minute.

From the above results, it can be seen that the system 1 and method are superb compared to the existing PPG based method (fiducial features) and is comparable with other biometric authentication systems.

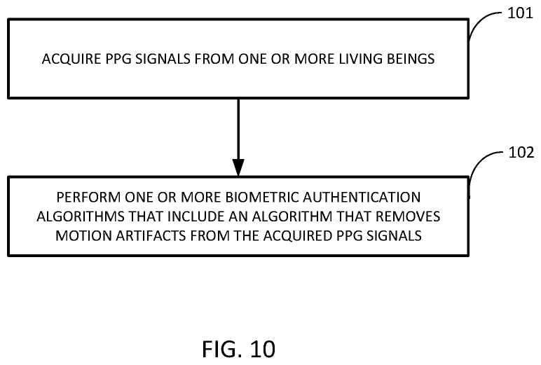

FIG. 10 is a flow diagram that illustrates the method for performing biometric authentication in accordance with a representative embodiment. As indicated by block 101, PPG signals from one or more living beings. As indicated by block 102, in processing logic configured to perform one or more biometric authentication algorithms, the acquired PPG signals are processed to perform biometric authentication of the living being(s), where at least one of the algorithm(s) is an MA removal algorithm that removes or at least mitigates MAs from the acquired PPG signals when performing biometric authentication.

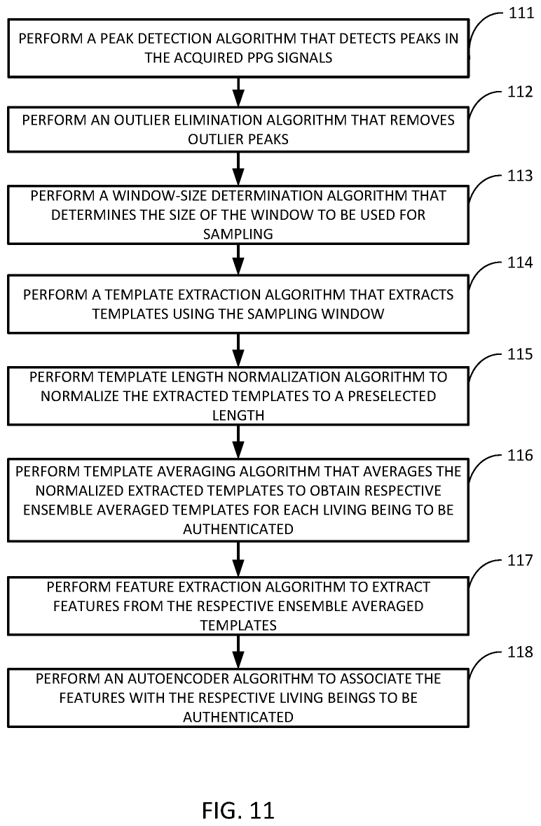

FIG. 11 is a flow diagram that illustrates the steps represented by block 102 of FIG. 10 in accordance with a representative embodiment. As indicated by block 111, a peak detection algorithm that detects peaks in the acquired PPG signals is performed after the MA removal algorithm has removed or at least mitigated MAs from the acquired PPG signals. As indicated by block 112, an outlier elimination algorithm is then performed that removes outlier peaks from the peaks detected by the peak detection algorithm. As indicated by block 113, a window-size determination algorithm is performed that determines a size of a window to be used to sample the acquired PPG signals' peak amplitudes. As indicated by block 114, a template extraction algorithm is performed that extracts templates from the acquired PPG signals using the window size determined by the window-size determination algorithm. As indicated above, the template extraction algorithm extracts a plurality of templates for each living being to be authenticated. As indicated by block 115, a template length normalization algorithm is performed that normalizes the extracted templates to a preselected length. As indicated by block 116, a template averaging algorithm is performed that averages the normalized extracted templates associated with each living being to be authenticated to obtain a respective ensemble averaged template for each respective living being to be authenticated.

After the ensemble averaged templates have been obtained, a feature extraction algorithm is performed that extracts at least one respective feature from each respective ensemble averaged template, as indicated by block 117. As indicated above, the feature extraction algorithm preferably comprises a multiwavelet decomposition algorithm. As indicated by block 118, an autoencoder algorithm is performed that processes the features to associate each feature with a respective living being to be authenticated. As indicated above, the autoencoder algorithm preferably comprises an MLP algorithm.

It should be noted that many variations may be made to the system and method within the scope of the inventive principles and concepts. For example, although FIG. 1 shows particular algorithms being used for particular purposes, other algorithms may be used for these purposes. In other words, the inventive principles and concepts are not limited to the representative embodiments described herein with reference to the figures. Although inventive principles and concepts have been illustrated and described in detail in the drawings and in the foregoing description, such illustration and description are to be considered illustrative or exemplary and not restrictive. The invention is not limited to the disclosed embodiments. Other variations to the disclosed embodiments can be understood and effected by those skilled in the art, from a study of the drawings, the disclosure, and the appended claims.

What is claimed is:

1. A system for performing biometric authentication comprising:
a signal acquisition device configured to acquire photoplethysmogram (PPG) signals from one or more living beings; and
processing logic configured to perform one or more algorithms that process the acquired PPG signals to perform biometric authentication of said one or more living beings, said one or more algorithms including a motion artifact (MA) removal algorithm that removes or at least mitigates MAs from the acquired PPG signals when performing biometric authentication.

2. The system of claim 1, wherein said one or more algorithms includes at least a peak detection algorithm that detects peaks in the acquired PPG signals after the MA removal algorithm has removed or at least mitigated MAs from the acquired PPG signals.

3. The system of claim 2, wherein said one or more algorithms includes at least an outlier elimination algorithm that removes outlier peaks from the peaks detected by the peak detection algorithm.

4. The system of claim 3, wherein said one or more algorithms includes at least a window-size determination algorithm that determines a size of a window to be used to sample the acquired PPG signals.

5. The system of claim 4, wherein said one or more algorithms includes at least a template extraction algorithm that extracts a template from the acquired PPG signals using the window size determined by the window-size determination algorithm.

6. The system of claim 5, wherein said one or more algorithms includes at least a template extraction algorithm that extracts a plurality of templates from the acquired PPG signals for each living being to be authenticated.

7. The system of claim 6, wherein said one or more algorithms includes at least a template length normalization algorithm that normalizes the extracted templates to a preselected length.

8. The system of claim 7, wherein said one or more algorithms includes a template averaging algorithm that averages the normalized extracted templates associated with each living being to be authenticated to obtain a respective ensemble averaged template for each respective living being to be authenticated.

9. The system of claim 8, wherein said one or more algorithms includes a feature extraction algorithm that extracts at least one respective feature from each respective ensemble averaged template.

10. The system of claim 9, wherein said one or more algorithms includes an autoencoder algorithm that processes said at least one respective extracted from the respective ensemble averaged template with a respective living being to be authenticated.

11. The system of claim 10, wherein the feature extraction algorithm comprises a multiwavelet decomposition algorithm.

12. The system of claim 11, wherein the autoencoder algorithm comprises a multilayer perceptron (MLP) algorithm.

13. A method for performing biometric authentication comprising:
acquiring photoplethysmogram (PPG) signals from one or more living beings; and
in processing logic configured to perform one or more algorithms, processing the acquired PPG signals to perform biometric authentication of said one or more living beings, said one or more algorithms including a motion artifact (MA) removal algorithm that removes or at least mitigates MAs from the acquired PPG signals when performing biometric authentication.

14. The method of claim 13, wherein said one or more algorithms includes at least a peak detection algorithm that detects peaks in the acquired PPG signals after the MA removal algorithm has removed or at least mitigated MAs from the acquired PPG signals.

15. The method of claim 14, wherein said one or more algorithms includes at least an outlier elimination algorithm that removes outlier peaks from the peaks detected by the peak detection algorithm.

16. The method of claim 15, wherein said one or more algorithms includes at least a window-size determination algorithm that determines a size of a window to be used to sample the acquired PPG signals.

17. The method of claim 16, wherein said one or more algorithms includes at least a template extraction algorithm that extracts a template from the acquired PPG signals using the window size determined by the window-size determination algorithm.

18. The method of claim 17, wherein said one or more algorithms includes at least a template extraction algorithm that extracts a plurality of templates from the acquired PPG signals for each living being to be authenticated.

19. The method of claim 18, wherein said one or more algorithms includes at least a template length normalization algorithm that normalizes the extracted templates to a preselected length.

20. The method of claim 19, wherein said one or more algorithms includes a template averaging algorithm that averages the normalized extracted templates associated with each living being to be authenticated to obtain a respective ensemble averaged template for each respective living being to be authenticated.

21. The method of claim 20, wherein said one or more algorithms includes a feature extraction algorithm that extracts a respective set of features from each respective ensemble averaged template.

22. The method of claim 21, wherein said one or more algorithms includes an autoencoder algorithm that processes the sets of features to associate each set of features with a respective living being to be authenticated.

23. The method of claim 22, wherein the feature extraction algorithm comprises a multiwavelet decomposition algorithm.

24. The method of claim 23, wherein the autoencoder algorithm comprises a multilayer perceptron (MLP) algorithm.
